(19) United States
(12) Patent Application Publication
(10) Pub. No.: US 2016/0324756 A1
(43) Pub. Date: Nov. 10, 2016

(54) USE OF POLYAMIDE POWDER IN A COSMETIC COMPOSITION FOR NAILS

(71) Applicant: ARKEMA FRANCE, Colombes (FR)

(72) Inventors: Helena CHEMINET, Bernay (FR); Stéfanie LAPORTE, Bernay (FR)

(73) Assignee: ARKEMA FRANCE, Colombes (FR)

(21) Appl. No.: 15/036,625

(22) PCT Filed: Nov. 13, 2014
(86) PCT No.: PCT/FR2014/052888

§ 371 Date: May 13, 2016

(30) Foreign Application Priority Data
Nov. 13, 2013 (FR) 1361079

(51) Int. Cl. A61K 8/88; A61Q 3/02; A61K 8/02

(52) U.S. Cl. CPC A61K 8/88; A61K 8/0241; A61Q 3/02; A61K 2800/412

(57) ABSTRACT

The use of polyamide powder particles in a cosmetic composition for the nails for improving the rate of drying, the mechanical strength, the matt appearance, the sliding appearance and/or the rough appearance of a layer of said composition on the nail, wherein: the volume-average diameter of the particles is within the range from 1 to 120 μm, the content of PA powder is within the range from 0.1 to 15% by weight, with regard to the total weight of the composition, the powder is added directly to said composition, the composition thus added is a varnish base, a makeup varnish for the nails, a finishing composition for the nails and/or a cosmetic care composition for the nails, said composition being pigmented or transparent.

The present invention relates to the field of compositions intended to repair and/or decorate nails, in particular cosmetic compositions for the nails, such as nail varnishes.

In addition to requirements conventionally desired for these nail compositions, such a rapid drying, long-term hold, mechanical strength, resistance to scratches, impact strength, or also effects of fortifying the nail with one or more vitamins, for example, formulators are looking for means for easily adjusting the matt/glossy appearance of the varnishes, the smooth or rough appearance, thus making it possible to obtain special effects, such as matt, rough, cracked, marked or “crocodile skin” effects, and the like, according to latest trends in fashion. The decoration of nails is truly becoming an art (“nail art”) which requires compositions of ever more innovative formulations, applicable with a brush, a pen or a stencil but also according to other more or less sophisticated methods, in particular by crosslinking under UV radiation.

It is thus an aim of the present invention to provide such cosmetic compositions contributing novel feels and/or novel visuals to the nail, which is what is understood by “special effects” within the meaning of the invention, while responding to the other requirements conventionally desired cited above.

Another aim of the present invention is to provide a simple process for the manufacture of such cosmetic compositions having special effects on the nail which exhibits as few stages as possible and which does not detrimentally affect the other cosmetic properties conventionally required for these nail compositions.

It has now been shown that it is possible to manufacture such nail cosmetic compositions, making it possible to obtain coatings having special effects on the nail, by virtue of the addition of specific polyamide powders, by varying in particular the particle size, the content and the type of polyamide powder used.

Polyamide powders are sometimes mentioned in the prior art as fillers for modifying the rheology of the liquid compositions of nail varnishes but the use of specific polyamide powders in order to obtain special effects on the nail varnish is not broached therein.

A subject matter of the present invention is thus the use of polyamide powder particles in a cosmetic composition for the nails for improving the rate of drying, the mechanical strength, the matt appearance, the sliding appearance and/or the rough appearance of a layer of said composition on the nail, in which:
the volume-average diameter of the particles is within the range from 1 to 120 μm,
the content of PA powder is within the range from 0.1 to 20%, preferably from 0.1 to 15%, by weight with regard to the total weight of the composition.
   The powder is added directly to said composition and optionally mixed, for example by simple stirring.

The volume-average diameter or measurement of the particle size of the powders in the present description, in particular in the examples and comparative examples, is carried out using a Coulter LS230 particle sizer. It makes it possible to obtain the particle size distribution of the powders, for which it is possible to determine the average diameter and the width of the distribution or the standard deviation of the distribution. The particle size distribution of the powders according to the invention is determined according to the normal techniques using a Coulter LS230 particle sizer from Beckman-Coulter. It is possible, from the particle size distribution, to determine the volume-average diameter with the logarithmic calculation method, version 2.11a. of the software, and also the standard deviation, which measures the narrowing of the distribution or the width of the distribution around the average diameter.

The composition thus additivated forms a varnish base, a makeup varnish for the nails, a finishing composition for the nails and/or a cosmetic care composition for the nails, said composition according to the invention being pigmented or transparent.
According to a preferred embodiment of the invention, said polyamide particles are chosen from polyamide particles, copolyamide particles and their blends.

Polyamide (homopolyamide or copolyamide) particles is understood to mean, within the meaning of the invention, the condensation products of lactams, of amino acids and/or of diacids with diamines and, as a general rule, any polymer formed by units connected to one another via amide groups. The particles according to the invention can also result from the copolymerization of lactam(s) with one or more lactone(s), resulting in a copolyesteramide, as described in the patent EP 1 172 396.

The term “monomer” in the present description of the copolyamides should be taken in the sense of “repeat unit”. The case where a repeat unit of the polyamide consists of the combination of a diacid with a diamine is special. It is considered that it is the combination of a diamine and of a diacid, that is to say the diamine.diacid pair (in equimolar amount), which corresponds to the monomer. This is explained by the fact that, individually, the diacid or the diamine is only a structural unit, which by itself alone cannot polymerize. In the case where the particles according to the invention comprise at least two different monomers, known as “comonomers”, that is to say at least one monomer and at least one comonomer (monomer other than the first monomer), they form a copolymer, such as a copolyamide, which is abbreviated to CoPA.

Mention may be made, as examples of lactams, of those which have from 3 to 12 carbon atoms on the main ring and which can be substituted. Mention may be made, for example, of β,β-dimethylpropiolactam, α,α-dimethylpropiolactam, amylolactam, caprolactam, capryllactam, oenantholactam, 2-pyrrolidone and lauryllactam.

Mention may be made, as examples of diacid (or dicarboxylic acid), of the acids having between 4 and 18 carbon atoms. Mention may be made, for example, of adipic acid, sebacic acid, azelaic acid, suberic acid, isophthalic acid, butanedioic acid, 1,4-cyclohexanedicarboxylic acid, terephthalic acid, the sodium or lithium salt of sulphoisophthalic acid, dimerized fatty acids (these dimerized fatty acids have a dimer content of at least 98% and are preferably hydrogenated) and dodecanedioic acid HOOC—(CH₂)₁₀—COOH.

Mention may be made, as examples of diamine, of aliphatic diamines having from 6 to 12 atoms; it can be arylic and/or saturated cyclic. Mention may be made, as examples, of hexamethylenediamine, piperazine, tetramethylenediamine, octamethylenediamine, decamethylenediamine, dodecamethylenediamine, 1,5-diaminohexane, 2,2,4-trimethyl-1,6-diaminohexane, polyoldiamines, isophoronediamine (IPD), methylpentamethylene-diamine (MPDM), bis(aminocyclohexyl)methane (BACM), bis(3-methyl-4-aminocyclohexyl)methane (BMACM), meta-xylyenediamine, bis(p-amino-cyclohexyl)methane and trimethylhexamethylenediamine.

Mention may be made, as examples of amino acid, of α,ω-amino acids, such as aminocaproic acid, 7-aminoheptanoic acid, 11-aminoundecanoic acid, 11-(n-heptylamino)undecanoic acid and 12-aminododecanoic acid.

Mention may be made, as examples of lactone, of caprolactone, valerolactone and butyrolactone.

The monomer(s) preferably used in the invention is or are chosen from lactams, such as, for example, lauryllactam, caprolactam, oenantholactam, capryllactam or their blends. Preferably, lauryllactam is used, alone or as a blend with caprolactam.

Preferably, the polyamide particles according to the invention comprise a content, as molar percentage, of polyamide 12 within the range extending from 50% to 100%, preferably from 80% to 100%.
Preferably, said polyamide-based powder particles are obtained at least in part by anionic polymerization, by seeding with an inorganic or organic filler, of lactam(s) and/or of lactone(s) in solution and/or in suspension in an organic liquid. Reference may be made, for example, to the processes described in documents EP 0 192 515 and FR 2 910 900. Advantageously, said porous particles in the present invention are spheroidal. They are preferably chosen from the powders sold under the Orgasol® brand name.

Alternatively, polyamide-based powder particles are obtained at least in part by hydrolytic polymerization, followed by grinding to give powders, generally of irregular shapes, without sharp edges. Such powders are preferably chosen from the powders sold under the Rilsan® brand name.

According to an advantageous embodiment of the present invention, the PA is chosen from PA11, PA12, PA10.10, the copolyamides comprising at least one of the following monomers: 6, 11, 12, 10.10, 10.12, 10.36, 6.10, 6.12 and 10.T, and their blends.

Another subject matter of the present invention is a cosmetic composition for the nails, comprising polyamide powder particles, characterized in that:
the volume-average diameter of the particles is within the range from 1 to 120 μm,
the content of PA powder is within the range from 0.1 to 15% by weight, with regard to the total weight of the composition,

and characterized in that it forms a film resistant to scratches after application to the nail and then drying.

According to an advantageous embodiment of the composition of the present invention,
the volume-average diameter of the particles is within the range from 5 to 20 μm,
the content of PA powder is within the range from 2 to 10%, preferably from 3 to 8%, by weight with regard to the total weight of the composition,

and characterized in that is forms a matt and sliding film after application to the nail and then drying.

Preferably, in this case, the PA comprises a content, as molar percentage, of polyamide 12 within the range extending from 50% to 100%, preferably from 80% to 100%.

According to another advantageous embodiment of the composition of the present invention,
the volume-average diameter of the particles is within the range from 80 to 120 μm, preferably from 100 to 120 μm,
the content of PA powder is within the range from 2 to 10%, preferably from 3 to 8%, by weight with regard to the total weight of the composition,

and said composition is characterized in that it forms a rough film after application to the nail and then drying.

Preferably, in this case, the PA comprises a content, as molar percentage, of polyamide 11, 10.10 or 10.12 within the range extending from 50% to 100%, preferably from 80% to 100%.

Another subject matter of the present invention is a cosmetic composition such as those defined above, characterized in that it is a varnish base, a makeup varnish for the nails, a finishing composition for the nails and/or a cosmetic care composition for the nails, it being possible for said composition according to the invention to be pigmented or transparent.

The present invention relates to the preparation of base coats (targeting the smoothing of the nail) as well as of top coats (targeting the protection of the varnish) or of varnishes (pigmented or transparent), whether these products are in the aqueous phase, solvent phase or 100% composed of solids and whether the varnish films are obtained by simple evaporation of the organic or aqueous solvent phase from a solution or dispersion of at least one film-forming polymer or by crosslinking under UV radiation.

The composition according to the invention includes one or more of the following main constituents commonly used in nail varnishes: film-forming agents, plasticizers, organic or aqueous solvents, diluents, pigments, pearlescent agents and/or coloring compounds, and thixotropic agents (or thixotropes).

The composition can additionally comprise at least one of the following compounds: inorganic or organic thickening agents, UV stabilizers, wetting agents, spreading agents, moisturizing agents, vitamins and/or fragrances.

### EXAMPLES

The examples below illustrate the present invention without limiting the scope thereof. In the examples, unless otherwise indicated, all the percentages and parts are expressed by weight.

The reference formulation is shown in the following table 1. The ingredients are added in the order shown by the lines of the table.

TABLE 1

| | % | Ingredients | Function | Supplier |
|---|---|---|---|---|
| a | 1000 | Tixogel LG-M | Thixotropic | Rockwood |
| | | | agent | |
| | 18 000 | Ethyl Acetate | Solvent | Brenntag |
| | 4000 | Isopropanol | Diluent | Brenntag |
| d | 13 000 | NC ECOCELL 70% | Nitrocellulose | Safic-Alcan |
| | | ds IPA | | |
| | 35 000 | Butyl Acetate | Solvent | Brenntag |
| | 4000 | Isopropanol | Diluent | Brenntag |
| | | Addition of (a) to (d) | | |
| e | 12 000 | Synolac 9614 BA 70 | Polyester resin | Arkema |
| f | 6000 | Citrofol BII | Plasticizer | Jungbunzlauer |
| g | 5000 | Butyl Acetate | Solvent | Brenntag |
| i | 2000 | Colorona Bordeaux | Pigment | Merck |

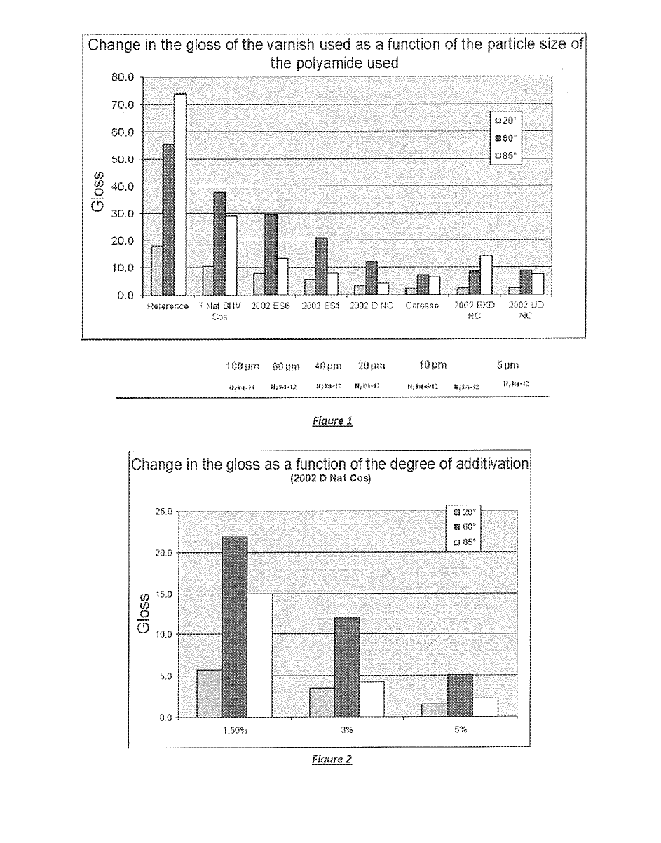

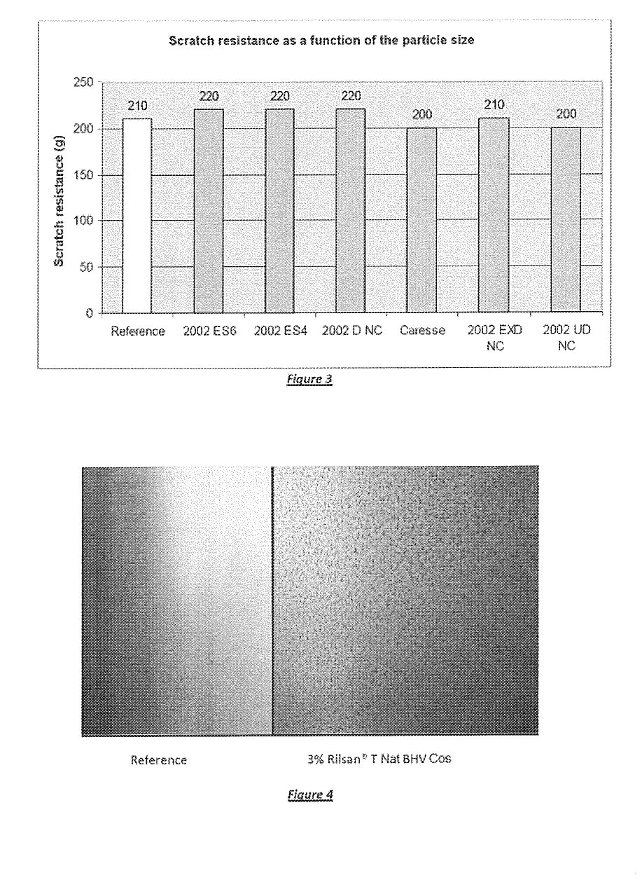

In the examples according to the invention, the reference formula of table 1 is additivated with each of the following polyamides (Orgasol grades sold by Arkema). In each case, the powder is simply added directly to the reference formula above, according to the contents, grades and particle sizes shown below. For the Orgasol® powders:
3% 2002 UD Nat Cos: PA12; 5 μm
3% 2002 EXD Nat Cos: PA12; 10 μm
1.5%, 3% or 5% 2002 D Nat Cos: PA12; 20 μm
3% Caresse: copolyamide 6/12; 10 μm
3% 2002 ES4 Nat 3: PA12; 40 μm
3% 2002 ES6 Nat 3: PA12; 60 μm
   For the Rilsan® powders:
3% T Nat BHV Cos: PA11; 104-114 μm.
   The coatings obtained after application of the same amount of varnish to the nail dry faster in the case of the compositions of the invention, by virtue of the addition of PA powder, than in the case of the reference composition without PA powder.
   The change in the gloss of a varnish, respectively as a function of the particle size and as a function of the degree of additivation of polyamide powder, is represented in the graph of FIG. 1 and in the graph of FIG. 2 respectively.
   It is observed that the matt appearance (opposite of gloss) increases when the particle size of the PA powder used according to the invention decreases (FIG. 1), and/or when the content of PA powder used according to the invention increases (FIG. 2).
   In the present invention, the glossy appearance (and thus, on the contrary, the matt appearance) is measured by means of a reflectometer, such as the Dr Lange REF0 glossmeter used here in the examples, for 3 angles of measurement: 20°, 60° and 85°, determined with respect to the normal of incidence perpendicular to the plane of the test specimen (sheet covered with a layer of dry varnish with a thickness of 50 μm). The measurement of the level of gloss corresponds to the percentage of light energy reflected at the opposite angle to the light ray emitted, with respect to the energy initially emitted. The glossmeter automatically carries out the emission, the measurement and the corresponding calculations.
   The scratch resistance of the varnish as a function of the particle size of the powder is represented in the graph of FIG. 3. This figure shows that the addition of PA powder to the reference varnish formula does not damage the scratch resistance of the coating obtained after application and drying of the composition on the nail, with respect to the coating obtained with the reference formula. Furthermore, for one and the same content of PA powder additivated to the reference formula, the more the particle size of the powder increases, the more the scratch resistance increases.
   FIG. 4 represents the roughness appearance (right-hand sheet) of the varnish obtained by addition of 3% of polyamide 11 powder with a volume-average diameter of 104-114 μm (Rilsan® T Nat BHV Cos) to the reference formulation.
   The following table 2 shows the static and dynamic coefficients of friction or sliding measured on coatings respectively obtained from the reference composition devoid of PA powder and from the reference composition additivated with 3% of Orgasol® 2002 EXD Nat Cos according to the invention. In the present invention, as well as for the examples, the coefficient of friction is measured using a Davenport device which moves, at a constant rate (15 cm/min), a disc (200 g) over a layer with a thickness of 50 μm of dry varnish on an aluminum sheet. The aluminum sheet coated with varnish was dried beforehand and stored in the open air (23° C., 50% humidity) for 7 days.
   The static coefficient of sliding (unitless) corresponds to the force necessary to start the disc moving (initial moving off):

(Value read (in g)×sensitivity (in this instance 2))/weight of the disc (200 g).

The dynamic coefficient of sliding (unitless) corresponds to the force necessary in order to keep the disc continuously moving after the start of sliding:

(Value read (in g)×sensitivity (in this instance 2))/weight of the disc (200 g).

A substantial improvement in the dynamic sliding and in the static sliding, by virtue of the addition of PA power according to the invention, is noted.

TABLE 2

| | Reference | Reference composition + |
|---|---|---|
| | composition | 3% 2002 EXD Nat Cos |
| Static coefficient of friction | 0.46 | 0.41 |
| Dynamic coefficient of friction | 0.36 | 0.27 |

1. A method of improving a rate of drying, mechanical strength, matt appearance, sliding appearance and/or rough appearance of a layer of a cosmetic composition on a nail, comprising:
adding polyamide (PA) powder particles to a cosmetic composition, wherein:
a volume-average diameter of the particles is within a range from 80 to 120 μm,
a content of PA powder is within a range from 0.1 to 15% by weight, with regard to a total weight of the cosmetic composition,
wherein the cosmetic composition thus additivated is a varnish base, a makeup varnish for the nails, a finishing composition for the nails and/or a cosmetic care composition for the nails, said composition being pigmented or transparent.

2. The method as claimed in claim 1, wherein the PA is chosen from copolyamides PA11, PA12, PA10.10, the copolyamides comprising at least one of the following monomers: 6, 11, 12, 10.10, 10.12, 10.36, 6.10, 6.12 and 10.T, and their blends.

3. A cosmetic composition for the nails, comprising polyamide (PA) powder particles, wherein:
a volume-average diameter of the particles is within a range from 80 to 120 μm,
a content of the PA powder is within a range from 0.1 to 15% by weight, with regard to a total weight of the cosmetic composition,
wherein the cosmetic composition is configured to form a film resistant to scratches after application to a nail and then drying.

4. A cosmetic composition for the nails, comprising polyamide (PA) powder particles, wherein:
the volume-average diameter of the particles is within the range from 5 to 20 μm,
the content of the PA powder is within the range from 2 to 10%, by weight with regard to the total weight of the cosmetic composition,
wherein the cosmetic composition is configured to form a matt and sliding film after application to a nail and then drying,
wherein the PA is chosen from copolyamides PA11, PA12, PA10.10, the copolyamides comprising at least one of the following monomers: 6, 11, 12, 10.10, 10.12, 10.36, 6.10, 6.12 and 10.T, and their blends.

5. The cosmetic composition as claimed in claim 4, wherein the PA comprises a content, as molar percentage, of polyamide 12 within a range extending from 50% to 100%.

6. The cosmetic composition as claimed in claim 3, wherein:
the content of PA powder is within the range from 2 to 10%, by weight with regard to the total weight of the composition,
wherein the cosmetic composition is configured to form a rough film after application to a nail and then drying.

7. The cosmetic composition as claimed in claim 6, wherein the PA comprises a content, as molar percentage, of polyamide 11, 10.10 or 10.12 within a range extending from 50% to 100%.

8. The cosmetic composition as claimed in claim 3, wherein the cosmetic composition is selected from the group consisting of a varnish base, a makeup varnish for the nails, a finishing composition for the nails and a cosmetic care composition for the nails,
said composition being pigmented or transparent.

9. The cosmetic composition as claimed in claim 8, wherein the cosmetic composition additionally includes one or more of constituents selected from the group consisting of film-forming agents, plasticizers, organic solvents, aqueous solvents, diluents, pigments, pearlescent agents, coloring compounds, and thixotropic agents.

10. The cosmetic composition as claimed in claim 8, wherein the cosmetic composition additionally includes at least one compound selected from the group consisting of inorganic or organic thickening agents, UV stabilizers, wetting agents, spreading agents, moisturizing agents, vitamins and fragrances.

11. A method of improving a rate of drying, mechanical strength, matt appearance, sliding appearance and/or rough appearance of a layer of a cosmetic composition on a nail, comprising:
adding polyamide (PA) powder particles to a cosmetic composition, wherein:
a volume-average diameter of the particles is within a range from 1 to 120 μm,
a content of PA powder is within a range from 0.1 to 15% by weight, with regard to a total weight of the cosmetic composition,
wherein the cosmetic composition thus additivated is a varnish base, a makeup varnish for the nails, a finishing composition for the nails and/or a cosmetic care composition for the nails, said composition being pigmented or transparent,
wherein the PA is chosen from copolyamides PA11, PA12, PA10.10, the copolyamides comprising at least one of the following monomers: 6, 11, 12, 10.10, 10.12, 10.36, 6.10, 6.12 and 10.T, and their blends.
